(19) United States
(12) Patent Application Publication
(10) Pub. No.: US 2026/0306120 A1
(43) Pub. Date: Oct. 8, 2026

(54) EMBOLIC RETRIEVAL SYSTEM AND METHOD

(71) Applicant: Triticum Ltd., Ramat-HaSharon (IL)

(72) Inventor: Doron Jacob BEN-AMI, Ramat-HaSharon (IL)

(73) Assignee: Triticum Ltd., Ramat-HaSharon (IL)

(21) Appl. No.: 19/481,547

(22) PCT Filed: May 29, 2024
(86) PCT No.: PCT/IL2024/050528

§ 371 Date: Nov. 5, 2025

Related U.S. Application Data
(60) Provisional application No. 63/469,460, filed on May 29, 2023.

(51) Int. Cl. A61B 17/221

(52) U.S. Cl. CPC A61B 17/221; A61B 2017/2212; A61B 2017/2215

(57) ABSTRACT

A system for emboli retrieval and methods of using same. The system includes a clot retrieval device including plurality of leaf-like extensions; and a collector configured for transitioning between a collapsed state when sequestered within an outer tube and an expanded state when advanced out of the outer tube, wherein the clot retrieval device is retrievable into the collector when the collector is in an expanded state. The system can optionally include a distal capturing device.

### RELATED APPLICATION/S

This application claims the benefit of priority of U.S. Provisional Patent Application No. 63/469,460 filed on 29 May 2023, the contents of which are incorporated herein by reference in their entirety.

### FIELD AND BACKGROUND OF THE INVENTION

The present invention relates to a system for retrieving emboli from a biological vessel. Specific embodiments of the present invention relate to a catheter system that includes an embolic retrieval device for dislodging and collecting embolic material, a collector for collecting the embolic retrieval device and distal capturing device for trapping the blood clot or a portion thereof during a retrieval procedure.

Occlusion of arterial vessels that supply blood to organs such as the brain can lead to morbidities such as stroke and death. Thus, restoring blood supply to organs by removing material occluding the vessel represents a first line therapy in such cases.

Two general approaches are utilized for mechanically removing thrombus material from a small blood vessel: a distal approach and a proximal approach.

In the distal approach, the distal end of the retrieval device (typically fitted with a distal basket or snare) is passed through the occlusion and positioned at a distal side thereof. The device is then pulled back (in a proximal direction) while the distal end engages the thrombus material.

In the proximal approach, the distal end of the retrieval device (fitted with a grasper or an aspirator) is brought into contact with the proximal side of the thrombus and the thrombus is then pulled proximally through the vasculature and finally removed from the body. One example of a device utilizing the proximal approach is the Penumbra device, manufactured by Penumbra Inc. and disclosed in EP 1799128.

A third and more commonly used approach involves opening a stent-like retrieval device inside the thrombus, compressing the thrombus material against the arterial wall and retrieving the device along with the compressed thrombus material.

Although these approaches can be used to at least partially remove thrombus material occluding an artery, such removal can oftentimes be associated with an increased risk of emboli fragmentation and release of thrombotic debris. In addition, current approaches have the added limitation of reduced efficacy in trapping and removing the entire emboli (J Neuroimaging 2021, Sep; 31(5): 912-924. doi: 10.1111/jon.1288).

As such, it would be highly advantageous to have a system capable of removing occlusive material from a biological vessel such as a blood vessel while being devoid of the above limitations.

### SUMMARY OF THE INVENTION

According to one aspect of the present invention there is provided a system for emboli retrieval comprising a clot retrieval device including plurality of leaf-like extensions; and a collector configured for transitioning between a collapsed state when sequestered within an outer tube and an expanded state when advanced out of the outer tube, wherein the clot retrieval device is retrievable into the collector when the collector is in an expanded state.

According to embodiments of the present invention the collector transitions to a funnel shape when advanced out of the outer tube.

According to embodiments of the present invention each of the plurality of leaf-like extensions is attached to a distal portion of an elongated body.

According to embodiments of the present invention the plurality of leaf-like extensions deploy radially outward when advanced out of the outer tube.

According to embodiments of the present invention the extensions are folded against the elongated body when advanced distally and deploy radially outward when the elongated body is pulled in a proximal direction.

According to embodiments of the present invention the leaf-like extensions are concave. According to embodiments of the present invention the leaf-like extensions include an inward curving distal tip.

According to embodiments of the present invention the collector is formed from a wire mesh or a laser cut or etched foil.

According to embodiments of the present invention the collector is coated with polymetric material, such as polyurethane, PTFE, silicone.

According to embodiments of the present invention the system further comprises a distal capturing device.

According to embodiments of the present invention the distal capturing device is attached to a distal end of the clot retrieval device or is deliverable therethrough.

According to embodiments of the present invention the distal capturing device is deliverable through a guidewire lumen of the elongated body.

According to embodiments of the present invention when expanded the distal capturing device is shaped as an umbrella, a parachute, a flower, a spiral or a ball. According to embodiments of the present invention the elongated body is deliverable through the collector.

The present invention successfully addresses the shortcomings of the presently known configurations by providing a device for effectively and non-traumatically retrieving an occlusion such as a thrombus from a biological vessel such as an artery.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEW OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-F illustrate the present system/kit showing the complete system (FIG. 1A) and its separate components including the retrieval device (FIG. 1E), the collector (FIG. 1D) and the distal capturing device (FIG. 1F) in a deployed state. Also shown are a guidewire (FIG. 1B) and the collector outer tube (FIG. 1C).

FIGS. 2A-E illustrate use of the present system in collecting embolic material from a blood vessel.

FIGS. 3A-H illustrate exemplary embodiments of a distal protection device that can be used in the present system.

FIG. 4 illustrates the forces applied on the collector.

### DESCRIPTION OF SPECIFIC EMBODIMENTS OF THE INVENTION

The present invention is of a system which can be used to retrieve occlusions from a biological vessel. The present invention is particularly useful for retrieving emboli from brain vessels.

The principles and operation of the present invention may be better understood with reference to the drawings and accompanying descriptions.

Before explaining at least one embodiment of the invention in detail, it is to be understood that the invention is not limited in its application to the details set forth in the following description. The invention is capable of other embodiments or of being practiced or carried out in various ways. Also, it is to be understood that the phraseology and terminology employed herein is for the purpose of description and should not be regarded as limiting.

In order to effectively clear an occlusion from an artery, thrombus material must be effectively penetrated, engaged/anchored, dislodged and retrieved from the vessel without releasing particles into circulation and while creating minimal irritation/damage to the vessel wall.

U.S. Pat. No. 9,987,027 to the present inventor discloses a catheter that is effective at penetrating, engaging, dislodging and retrieving thrombus material while minimizing damage to the vessel wall.

While experimenting with several device prototypes, the present inventor realized that while the catheter design is highly effective in dislodging and collecting embolic material from an occluded artery, in some cases, when pulled back into larger vasculature (e.g., carotid artery), engagement between the embolic material and the retrieval device might be compromised due to the increase in vessel diameter and as a result a decrease in the inward radial forces acting on the device.

The directionality of the leaf-like projections of the retrieval device facilitates penetration into the occluding blood clot and when pulled backward, resistance from the clot opens the projections, resulting in scooping of the clot material.

Once the blood clot is retrieved, the catheter is pulled back into the collector (funnel) which is designed to contact the vessel walls at its distal opening such that the leaf-like projections and clot are centralized and collected into the funnel without clot fragmentation or loss.

The distal capturing device ensures that any clot fragments that may form are captured and collected into the funnel when the distal capturing device is pulled backwards.

Thus, according to one aspect of the present invention there is provided a system and kit for removing (clearing and optionally retrieving) occlusions in a biological vessel. As used herein, the phrase “biological vessel” refers to any vessel capable of supporting flow of a biological material. The vessel can be a natural vessel or a synthetic vessel implanted in a body. Examples of vessels include blood vessels such as veins or arteries, lymphatic vessels, urinary system vessels such as the urethra or ureters, seminal vessels, saliva ducts, bile ducts and more. Occlusions are any flow limiting blockages (emboli) in the vessel which are caused by local buildup of material, examples include atherosclerotic emboli, migrating blood clots, biological stones, foreign bodies or the like.

The present system includes three components that function in unison to collect and retrieve embolic material from the biological vessel. These components include a retrieval device having an elongated body for delivering a plurality of leaf-like extensions arranged around a distal portion of the elongated body into the biological vessel.

When advanced out of a delivery tube, the leaf-like extensions deploy outward and are positioned at a rearward (proximal direction) angle with respect to the elongated body. When the extensions are advanced passed occlusive material and pulled proximally, their leaf-like shape (with inner concavity) scoop-up the occlusive material.

The retrieval device can be configured as a catheter for use with a guidewire (GW) in clearing material from a blood vessel. When configured as a catheter, the elongated body can include a longitudinal lumen sized for accepting a guidewire (e.g., 0.014″, 0.018″ or 0.035″ or other guidewires). The lumen can be configured for use with over-the-wire, or rapid exchange systems.

The elongated body of the retrieval device can be 10 to 200 cm in length with a width/diameter of 0.05-50 mm when in closed configuration (suitable for delivery within a 0.1-30 F sheath. The elongated body is preferably shaped as shaft (rod or tube) and is fabricated from any bio-compatible material, including, for example, alloys such as stainless steel, Nitinol or polymers such as Polyimide (PI), Polyether Block Amide (PEBA)-Pebax. The elongated body is preferably axially rigid in order to facilitate lodging of the distal portion (carrying the extensions) into the occlusion and yet flexible enough to facilitate navigation through torturous vessels while ensuring safety (e.g., blood vessels in the brain). Rigidity of the elongated body (catheter) is same range as catheters commonly used for navigating biological vessels such as blood vessels.

The extensions deploy radially outward, preferably at an angle (of 0 -60 degrees) towards the proximal end of the elongated body. The extensions can be of any shape (rectangle, triangle, oval, polyangular-shaped, spiral, or a combination of several shapes including simple or complex shapes with fractal characteristics) and of any profile (round, oval, rectangle). The extensions can be directly connected to the elongated device body, or connected thereto through a joint element (e.g., stem).

The axial rigidity of the stem portion of the extension can be preferably anywhere from 0.1-100 grams (e.g., 10-90, 20-80, 30-70, 40-60) or more depending on the occlusion location, occlusion type and size, extension structure and material the stem is constructed from. The axial rigidity of the extension can be anywhere from 0.0-50 grams (e.g., 5-40, 10-30, 20-25) or more depending on the occlusion location, occlusion type and size and the structure and material the extension is constructed from.

The extensions and optionally stems are preferably elastically deformable and fabricated from elastomeric material such as thermoplastic elastomers (TPEs), silicone, other plastics or metal alloys such as Nitinol. Elasticity is selected such that when the device is advanced distally into an occlusion (thrombus) within the biological vessel, the extensions fold against the elongated body due to the forces exerted by the occlusion/thrombus mass. This enables the extensions to penetrate an occlusion (e.g. thrombus) in the vessel without crossing or deploying distally outside to the thrombus mass and lodge therein. When the device is pulled in a proximal direction, the extensions deploy outward (to the angle set by the stems or the vessel wall limitation) due to the drag forces exerted by the occlusion (thrombus) mass thereby enabling the device to engage/anchor to the occlusion material, dislodge it from the vessel wall and remove it.

Typical dimensions for the extensions can be 0.2-30 mm in length, 0.05-20 mm in width, 0.03-3 mm in thickness, with a single side surface area of 0.01-600 mm².

The stems portions can be 0.1-20 mm in length, 0.02-20 mm in width, 0.03-3 mm in thickness.

Any number of extensions can be carried on the elongated body depending on the biological vessel, occlusion size and type and function of the device. A typical number of extensions can range from 1-20 or more. The extensions can be carried as pair, triplets etc., on a fixed or swiveling joint.

The internal surface (facing towards the elongated body) of the extensions is preferably concave in order to increase the surface area thereof and the drag/resistance force exerted on the internal surface by the thrombus mass. Such a concave configuration also increases the ability of the extensions to collect (scoop) the occlusion material. The exterior surface of the extensions is preferably convex to facilitate delivery within the vessel and lodging of the extensions into the occlusion while folded in a “close configuration” (arrow like) due to the drag forces exerted on the extensions by the occlusion material when the extensions are advanced into the occlusion. Although such a configuration is preferred, internal and external surfaces having alternative contours (e.g., flat on both sides) are also envisaged herein.

Each extension can also fold in half lengthwise to further improve penetration into the occlusion material. Such folding can occur during use, in accordance with the mechanical forces exerted upon the extensions by the occlusion material and the vessel wall.

The distal portion (tip) of the extension is preferably curved inward in order to minimize trauma/damage to the vessel when the device is navigated within the blood vessels. To further decrease trauma and irritation to the vessel wall, the tips can be fabricated from a very soft material (softer than the rest of the leaf-like structure).

The inward curving tips can also facilitate hooking of the extensions into the occlusion material.

The inner (and optionally outer) surface of the extensions can include surface mounted projections arranged as an array of specific size and distribution in order to enable the projections to engage a repeating structure on the surface of the occlusion.

In the case of a thrombus, this repeating structure is the fibrin mesh component of the thrombus.

The present system/kit also includes a collector shaped as a funnel (when deployed) that functions in collecting the retrieval device along with the occlusive material. The funnel is positioned within an outer tube and is deployed (radially outward) when advanced out of the outer tube (or when unsheated by pulling the tube backward). The funnel includes a distal opening for accepting the retrieval device and occlusive material. The distal opening can be closed via a pull wire or any other mechanism known in the art.

The funnel is attached to an inner tube having a lumen for accepting the elongated body and distal extensions of the retrieval device. Such a lumen can also carry the pull wire used for optional closure of the distal opening.

The funnel can be fabricated from a cut tube or wire mesh (Nitinol or stainless steel—round or flat wires) and can have the following dimensions: length: 10-100 mm, diameter: 1-50 mm. The diameter range of the round wires between 0.01-0.5mm; or flat wires 0.01-0.5 mm thick; braid density between 50-110 PPI (Picks Per Inch). The funnel can be coated/covered with polymeric material, such as polyurethane, PTFE, silicone and the like.

A vacuum source (e.g., pump, syringe) can be connected to the system and used to apply suction to the internal volume of the coated funnel to assist in collection of the clot retrieval device.

When sequestered within the outer tube, the funnel assumes a tube-like shape that is restricted to the size and shape of the lumen of the outer tube. The funnel is deployed by advancing it out of the outer tube using the inner tube that is attached to the proximal end of the funnel. Once advanced out, the funnel forms the funnel-like shape with the distal opening restricted to a size dictated by the diameter of the biological vessel lumen.

The system/kit also includes a distal capturing device that functions as another safety measure to ensure full clot retrieval. The retrieval device and capturing device are positioned distally to the clot. The capturing device serves as a barrier to any fragmented or dislodged material. The distal capturing device can be delivered through the lumen (e.g., 0.017 inch) of the elongated body or through another dedicated lumen of the elongated body and includes a trap attached to a wire/rod for advancing the collector through the GW lumen

Once deployed, the trap of the distal capturing device assumes any one of several shapes including but not limited to, pigtail, ball-like shape, flower-like shape, parachute-like shape, net-shape, spiral or any other shape suitable for capturing embolic fragments.

The distal capturing device can be pulled into the funnel completely or to cap the funnel distal opening.

The present system/kit can be used as follows. The outer tube including the collector and retrieval device can be navigated over the wire to a site proximately to the occlusion. The retrieval device can then be advanced out of the collector and into and passed the occlusion. The GW can then be removed and a distal capturing device is advanced through the guidewire lumen and positioned at a site distal to the occlusion. The collector can then be deployed and the retrieval device can then be pulled proximally to retrieve the occlusive material while the capturing device is in position to capture any upstream fragments. The retrieval device and distal capturing device can then be pulled proximally into the collector and the distal capturing device can be pulled proximally to cover the distal opening of the collector. The entire system can then be pulled into the outer tube and out of the body.

The distal tip of the leaf-like projections can be curved inward in order to facilitate centralization of the retrieval device into the collector. Centralization can also be achieved by adding, at the proximal end, an olive-like structure, or a leaf-like structure (in a direction opposite to that of the distal end leaf-like structure) in order to centralize the proximal portion of the device when pulled in. A proximal leaf-like structure can include projections (leaves) that are designed for flip over when pulled into the collector. Coating of the mesh funnel and smaller mesh pores (0.01-0.5 mm wide) as well as various mesh wire profiles can be used to reduce friction between the projections and the inner surface of the collector.

Since the present device can be used in both large vessels as well as small vessels, the collector is designed to maintain a relatively low radial outer force while fully/partially deployed in the relatively small (distal) vasculature while being capable of full radial deployment in the larger proximal vasculature. This is unlike stent retrievers that require high radial forces for function.

Such functionality can be achieved by configuring the braid geometry of the collector to exert a relatively low radial forces in smaller vessels (e.g., 0.5-3.0 mm internal diameter ID) and yet provide full deployment at larger vessel sizes (e.g., 3.0-7.0 mm ID).

The radial forces that the collector applies are relatively lower when compared to stent retrievers and therefore when partially open in the small vessels or fully open in the large vessels, its function is only to fill the vessel space while exerting a minimal force on the internal walls of the vessel.

In contrast to laser-cut stent retrievers that are fabricated from a metal tube and as such, apply radial forces along the entire device length, a braided device can be configured with multiple parameters to control the radial forces applied to the vessel wall. For example, wire characteristics (flat vs round, thickness, material type), wire number and the braid configuration can be selected to apply a variable force along device length.

The collector is designed to allow clot fragments to enter the capturing device and to capture these fragments while applying minimal radial force on the blood vessel.

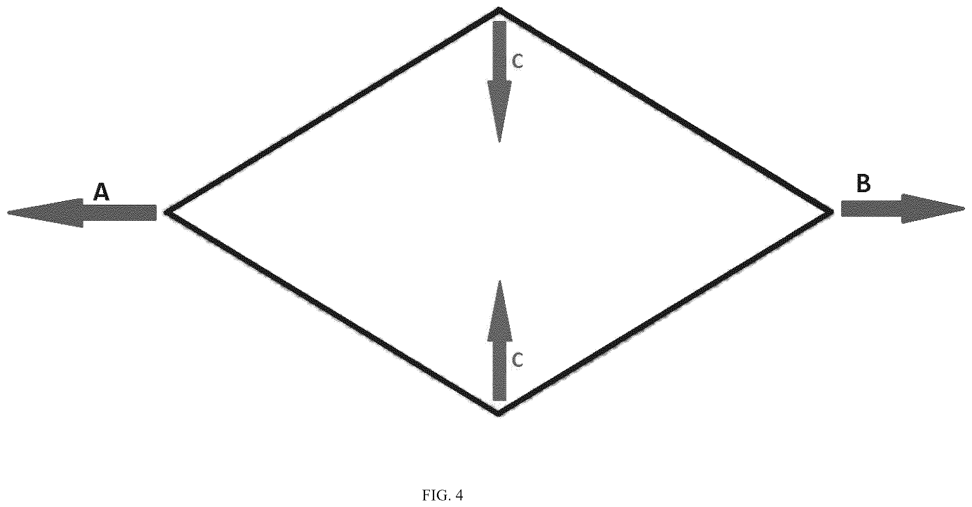

To that end, the braid configuration includes relatively large “windows” (openings) on its proximal side (<4mm) in order to catch clot fragments and relatively small windows (<1.5 mm) on its distal side in order to catch and hold clot fragments. The braid is shaped as two cones connected at their base forming a diamond-like cross section. This design allows two main functions:
(a) Minimal direct contact with the blood vessel (only at its largest diameter at middle)—this allows the collector to apply minimal radial forces on the vessel wall while being pulled back; and
(b) The geometric shape reduces the force applied on the vessel wall while the collector is being pulled back-as is shown in FIG. 4, the force applied on the collector by the clots captured thereby (B) and the flow of blood is opposite to the force applied by the physician (A); these opposing forces generate an inward radial force that reduces the friction between the collector and the vessel wall (c).

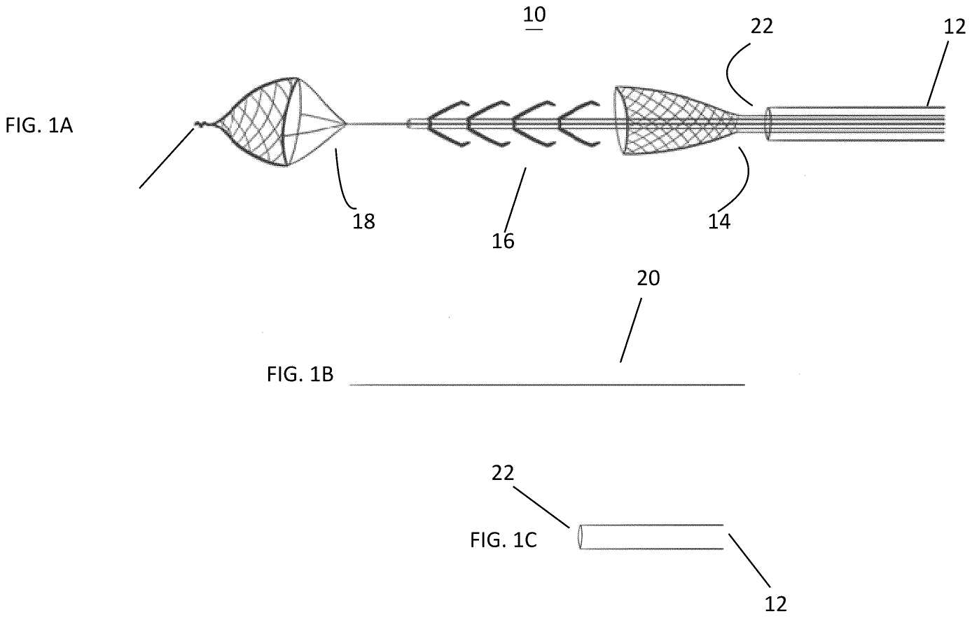

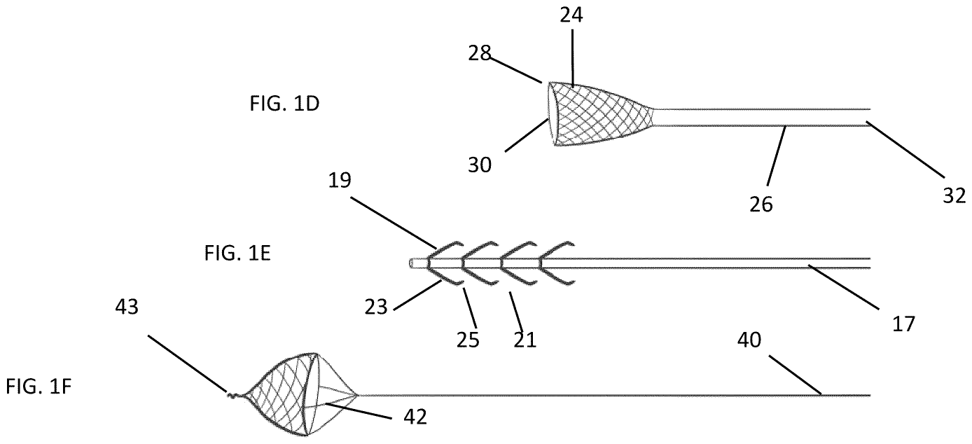

Referring now to the drawings, FIGS. 1A-3H illustrate the present system which is referred to herein as system 10. System 10 (assembled in FIG. 1A) can be provided as a kit that includes some or all of the components shown in FIGS. 1B-F.

System 10 includes an outer tube 12 (sheath) for housing a collector 14, a retrieval device 16 and a distal capturing device 18 (e.g., in a coaxial layout). System 10 can be delivered and deployed over a wire 20 as is further described hereinunder.

When used for emboli retrieval in brain blood vessels, outer tube 12 can be 90 cm in length, 8 F (0.105 inch) mm in outer diameter and 6.7 F (0.088 inch) in inner diameter (diameter of inner lumen 22). Outer tube 12 can be fabricated from a polymer using well know approaches.

Inner lumen 22 of outer tube 12 can be used to deliver collector 14 which includes a funnel 24 attached to a tube 26. Funnel can be 10-30 mm in length, 0.017-0.027 inch in diameter when collapsed and 2-6 mm in diameter at distal end 28 when expanded. Funnel 24 can be fabricated from 0.055-0.075 mm wires (Nitinol or combination of Nitinol and other metals) that are braided at an angle between 105-135 degrees and have a cell size of 0.3-0.8 mm. Funnel 24 can self-expand when advanced out of outer tube 12 or it can be manually expanded using well know approaches (e.g., pull wire). Opening 30 at distal end 28 is limited in size to the vessel inner diameter. Tube 26 can be 100-125 cm in length 0.75-0.85 inch in outer diameter and 0.58-0.72 inch in inner diameter (diameter of inner lumen 32). Inner lumen 32 can be used to deliver wire 20 (e.g., GW) or distal capturing device 18.

Retrieval device 16 includes a tube 17 having a plurality of extensions 19 attached to a distal portion 21. Tube 17 can be 150-160 cm in length, 0.028-0.034 inch in outer diameter and 0.017-0.021 inch in inner diameter (diameter of inner lumen 27-27 is missing in the drawings)

Each extension 19 can include a leaf-like body 23 (3-6 mm in length, 1-3 mm in width at middle with an inner concavity) and an inward curving end 25. Extensions 19 are arranged around distal portion 21 in opposing pairs, staggered (to form a spiral) or any other suitable configuration. Inner lumen 27 can be used to deliver GW 20 or capturing device 18.

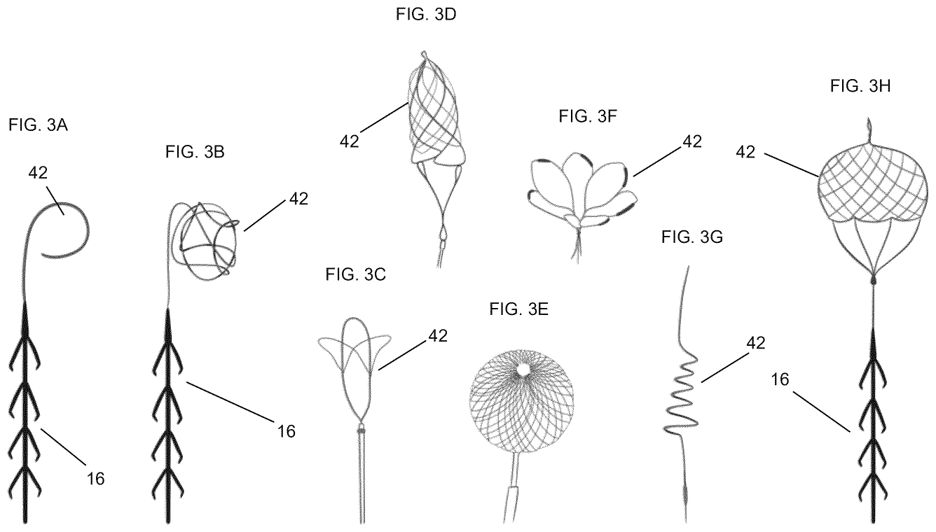

Distal capturing device 18 can include a wire/rod 40 attached to a trap 42. As is shown on FIGS. 3A-H, trap 42 can be any one of several trap 42 designs including, but not limited to a loop (FIG. 3A), a ball/nest (FIG. 3B, E), a flower (FIG. 3C, F), a cone (FIG. 3D), a spiral (FIG. 3G) and a parachute (FIG. 3H). Trap 42 can include a soft tip 43 at its distal end.

Trap 42 functions in capturing embolic fragments resulting from retrieval of occlusive material. Trap 42 can be designed for self-deployment when advanced out of tube 17 through its inner lumen (17). To that end, trap 42 can be fabricated from a wire or wires woven into any shape suitable for use as a trap. Trap 42 can also include radiopaque markers 44—missing 44 in the drawing (FIG. 3F) for correct positioning with respect to retrieval device 16 and/or the occlusion.

As is mentioned herein, the present system/kit can be used to retrieve occlusive material from any vessel.

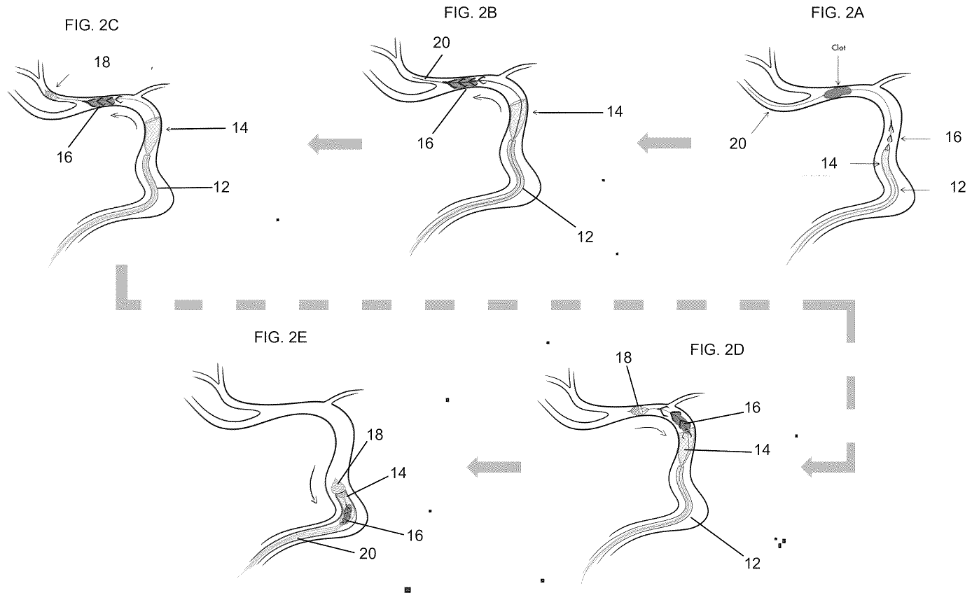

FIGS. 2A-E illustrate use of system 10 in a brain artery of the middle cerebral artery.

A femoral or radial artery is punctured using a Seldinger's procedure, the carotid artery is catheterized, and a long sheath is advanced to the internal carotid artery at the side of the occlusion. System 10 is advanced co-axially on a guide wire 20 (FIG. 2A), collector 14 is advanced and to the distal tip of the sheath, while retrieval device 16 is advanced further into the occluding thrombus (FIG. 2B). Guidewire 20 is exchanged with a distal capturing device 18 that is advance through the microcatheter of retrieval device 16 and is deployed distally to the occlusion (FIG. 2C).

Collector 14 is deployed through sheath 12 at the carotid artery and retrieval device 16 along with capturing device 18 are pulled backward into collector 14. Aspiration is optionally applied through collector 14 and sheath 12. Alternatively, a balloon guiding catheter (a guiding catheter with proximal balloon) can be used for retrieval instead of sheath 12.

Collector 14, retrieval device 16 and capturing device 18 (with trapped embolic material) are pulled back into sheath 12 of balloon guiding catheter and removed from patient's body.

As used herein the term “about” refers to ±10%.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A system for emboli retrieval comprising:
(a) a clot retrieval device including plurality of leaf-like extensions; and
(b) a collector configured for transitioning between a collapsed state when sequestered within an outer tube and an expanded state when advanced out of said outer tube, wherein said clot retrieval device is retrievable into said collector when said collector is in an expanded state.

2. The system of claim 1, wherein said collector transitions to a funnel shape when advanced out of said outer tube.

3. The system of claim 1, wherein said plurality of leaf-like extensions are attached to a distal portion of an elongated body.

4. The system of claim 1, wherein said plurality of leaf-like extensions deploy radially outward when advanced out of said outer tube.

5. The system of claim 4, wherein said extensions are folded against said elongated body when advanced distally and deploy radially outward when said elongated body is pulled in a proximal direction.

6. The system of claim 1, wherein said leaf-like extensions are concave.

7. The system of claim 1, wherein said leaf-like extensions include an inward curving distal tip.

8. The system of claim 1, wherein said collector is formed from a wire mesh or a laser cut or etched foil.

9. The system of claim 8, wherein said collector is coated with polymetric material, such as polyurethane, PTFE, silicone.

10. The system of claim 3, further comprising a distal capturing device.

11. The system of claim 10, wherein said distal capturing device is attached to a distal end of said clot retrieval device or is deliverable therethrough.

12. The system of claim 11, wherein said distal capturing device is deliverable through a guidewire lumen of said elongated body.

13. The system of claim 10, wherein when expanded said distal capturing device is shaped as an umbrella, a parachute, a flower, a spiral or a ball.

14. The system of claim 3, wherein said elongated body is deliverable through said collector.
